(19) United States
(12) Patent Application Publication
(10) Pub. No.: US 2015/0328437 A1
(43) Pub. Date: Nov. 19, 2015

(54) RECTAL TUBE SECUREMENT DEVICE

(71) Applicant: Noureyah Rageh, Dearborn, MI (US)

(72) Inventor: Noureyah Rageh, Dearborn, MI (US)

(21) Appl. No.: 14/716,679

(22) Filed: May 19, 2015

Related U.S. Application Data
(60) Provisional application No. 62/000,224, filed on May 19, 2014.

(51) Int. Cl. A61M 25/02; A61F 5/449

(52) U.S. Cl. CPC A61M 25/02; A61F 5/449; A61M 2025/0213; A61M 2025/028; A61M 2025/0266; A61M 2202/068; A61M 2205/02

(57) ABSTRACT

A securement device for securing a rectal tube is disclosed. The securement device has a hollow cylindrical tube sized to accommodate a rectal tube, the hollow cylindrical tube having an opening at a first end and at a second end and a split running in an axial direction from the first end to the second end that allows a rectal tube to be snapped into the hollow cylindrical tube. The securement device also has one or more flat supports connected to the second end of the hollow cylindrical tube, each coated with an adhesive that can secure the device to the skin of a patient. Each flat support has a limited length connection with the hollow cylindrical tube, such that at least a portion of the circumference of the second end of the hollow cylindrical tube is not connected to the flat support.

### CROSS-REFERENCE TO RELATED APPLICATION

The present application claims the benefit of priority of U.S. Provisional Application No. 62/000,224, filed May 19, 2014. The entire text of the priority provisional application is incorporated herein by reference in its entirety.

### FIELD OF THE INVENTION

This disclosure relates generally to a rectal tube anchor and method of use for securing a rectal tube within the anus of a patient suffering from fecal incontinence so as to prevent the rectal tube from becoming dislodged from the patient.

### BACKGROUND OF THE INVENTION

Patients with fecal incontinence may require a rectal tube, sometimes referred to as a rectal trumpet, to be inserted into their anus. A rectal tube may become dislodged from the patient if the patient is turned in a hospital bed, thus requiring that the rectal tube be reinserted by a healthcare professional. Further, unsanitary conditions may result from the rectal tube becoming dislodged. If an accident occurs and the rectal tube is dislodged, the patient may become soiled, thus requiring the patient to be cleaned and the patient's bedding to be replaced. This can be time consuming and cumbersome for healthcare professionals, and can be uncomfortable and embarrassing for the patient. Further, reinserting the rectal tube can cause the patient pain and discomfort.

Devices are known in the prior art that relate to medical device attachments. Some devices include means for securing a medical device to the skin of a patient by means of a skin-friendly adhesive. Other devices relate to ostomy faceplates having an adhesive wafer for securement to a user.

### SUMMARY OF THE INVENTION

Embodiments within the scope of the present disclosure are directed to a securement device for use with a rectal tube that is adapted to maintain a rectal tube in position on a patient. In embodiments within the scope of the present disclosure, the securement device includes a hollow cylindrical tube having an opening at a first end and at a second end. The hollow cylindrical tube is made from a flexible non-latex plastic and is split at one location in the axial direction from the first end to the second end so that a rectal tube can be snapped into the hollow cylindrical tube through the split. The split in the hollow cylindrical tube also enables the securement device to accommodate a range of different sized rectal tubes. In some embodiments within the scope of the present disclosure, the interior portion of the hollow cylindrical tube is coated with an adhesive that sticks to an outer side of a rectal tube once it has been inserted, attaching the securement device to the rectal tube. In other embodiments, in addition to or instead of an adhesive on the interior portion of the hollow cylindrical tube, an adhesive flap can be secured to the hollow cylindrical tube on one side of the split. Once the rectal tube has been snapped into the hollow cylindrical tube through the split, the adhesive flap may be wrapped around the exterior portion of the hollow cylindrical tube across the split and adhered to the hollow cylindrical tube on the opposite side of the split from which it originated, thus compressing the interior of the hollow cylindrical tube onto the exterior of the rectal tube to help secure the hollow cylindrical tube to the rectal tube.

Flat supports are connected to the second end of the hollow cylindrical tube and extend outward therefrom. Each flat support has a proximal surface with an adhesive that is secured to the skin of the patient in order to keep the securement device, and consequently the rectal tube, in place. In some embodiments within the scope of the present disclosure, two flat supports are connected to the second end of the cylindrical tube extending radially in different directions. In other embodiments within the scope of the present disclosure, one flat support or more than two flat supports may be connected to the second end of the cylindrical tube. Even when a rectal tube is properly secured onto a patient, some stool may leak around the exterior portion of the rectal tube. If the connections between the flat supports and the second end of the cylindrical tube extend all the way around the circumference of the cylindrical tube, stool that has leaked around the outside of the rectal tube will become trapped underneath the flat supports, causing an unsanitary and potentially uncomfortable situation for the patient. To avoid this, some embodiments within the scope of the present disclosure have flat supports that are bow shaped. The bow shape allows the portion of the flat support connected to the second end of the hollow cylindrical tube to be fairly small, such that leaked stool will not become trapped underneath the flat support but can instead escape from around the edges of the second end of the hollow cylindrical tube that are not connected to a flat support and be wiped away. The angled side edges of a bow shaped flat support allow the flat support to have a fairly large surface area, despite the small portion connected to the second end of the hollow cylindrical tube. The surface area of the bow shaped flat support is adhered to a patient's skin and helps to keep the securement device, and consequently the rectal tube, in place.

In use, a healthcare professional inserts a rectal tube into a patient's anus. The securement device is secured to the rectal tube by snapping a portion of the rectal tube that is near the patient's anus through the split into the hollow cylindrical tube. If the hollow cylindrical tube has adhesive on its interior portion, any removable protective backing covering the adhesive should be removed prior to snapping the rectal tube into the hollow cylindrical tube so that the interior portion of the hollow cylindrical tube will immediately adhere to the outer side of the rectal tube. If the hollow cylindrical tube has an adhesive flap, any removable protective backing covering the adhesive on the flap is removed and the adhesive flap is then wrapped around the exterior portion of the hollow cylindrical tube across the split and adhered to the hollow cylindrical tube on the opposite side of the split from which it originated. Any removable protective backing of the adhesive on the flat supports is then removed, the patient's skin may optionally be wiped with a no-sting barrier wipe to enhance the ability of adhesive to stick to the patient's skin, and the flat supports are adhered to the skin of the patient's buttocks. In this way, the rectal tube is secured to the patient and is not easily dislodged.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 illustrates a perspective view of an embodiment of a securement device securing a rectal tube within the anus of a patient by securing a hollow cylindrical tube having a first end and a second end around a portion of the rectal tube near the anus of the patient, the hollow cylindrical tube having flat supports extending from the second end of the hollow cylindrical tube that are adhered onto the skin of the patient's buttocks.

FIG. 2 illustrates a perspective view of the hollow cylindrical tube of the securement device depicted in FIG. 1, which has a split in the axial direction and an adhesive flap secured to the hollow cylindrical tube on one side of the split, as a removable protective backing of the adhesive flap is removed.

FIG. 3 illustrates a perspective view of the hollow cylindrical tube of the securement device depicted in FIGS. 1 and 2 as the adhesive flap is wrapped around the exterior portion of the hollow cylindrical tube and adhered to the hollow cylindrical tube on the opposite side of the split.

FIG. 4 illustrates a perspective view of the pair of bow shaped flat supports extending from the second end of the hollow cylindrical tube of the securement device depicted in FIGS. 1-3 as a removable protective backing of one bow shaped flat supports is removed.

### DETAILED DESCRIPTION OF THE INVENTION

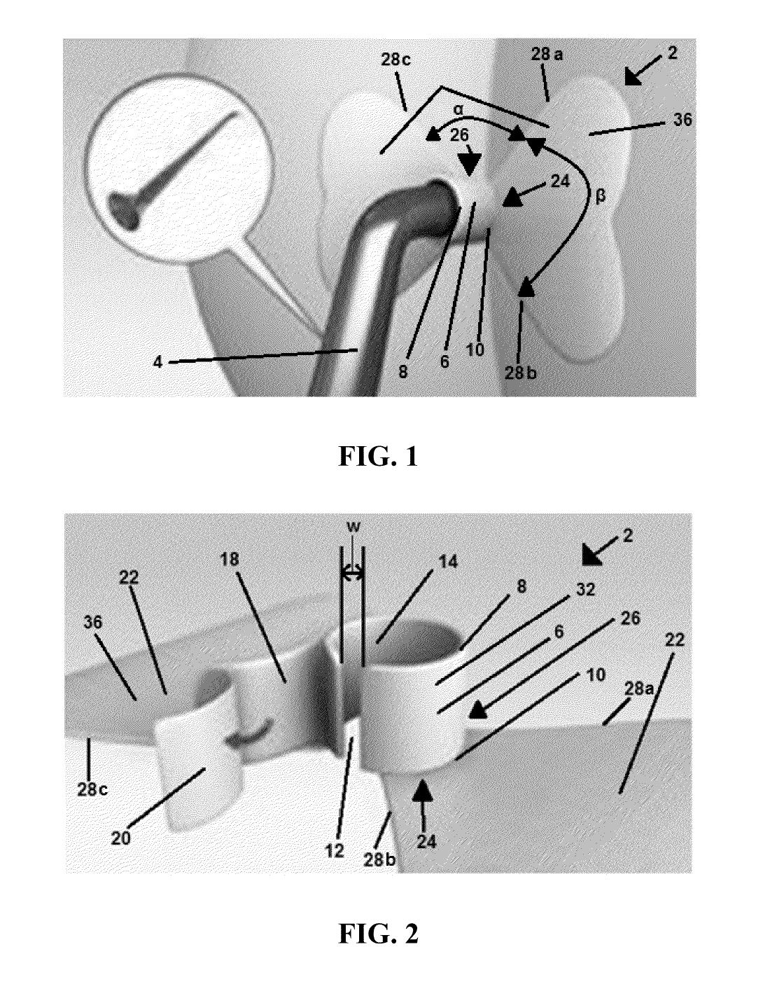

Referring now to the figures in detail, FIG. 1 illustrates an exemplary securement device 2 securing a rectal tube 4 within the anus of a patient. The exemplary securement device 2 has a hollow cylindrical tube 6 and two flat supports 22. The hollow cylindrical tube 6 has an interior portion 14 and an exterior portion 32 and is secured to the rectal tube 4 by a fixation device. As described in greater detail below, the fixation device may be an adhesive flap 18 wrapped around the exterior portion 32 of the hollow cylindrical tube 6, an adhesive coating on the interior portion 14 of the hollow cylindrical tube 6, or another fixation device known in the art. The flat supports 22 are spaced circumferentially around the hollow cylindrical tube 6 and are connected to the hollow cylindrical tube 6 by an adhesive, glue, welding, or by being integrally formed with the hollow cylindrical tube 6. The flat supports 22 are adhered to the skin of the patient's buttocks to keep the securement device 2 in place.

The hollow cylindrical tube 6 has a first end 8 and a second end 10. The hollow cylindrical tube 6 is preferably made from a flexible plastic. In some embodiments, the hollow cylindrical tube 6 can be made from a non-latex material in order to avoid causing complications for patients with a latex allergy. As shown in FIG. 2, the hollow cylindrical tube 6 has a split 12 running in the axial direction from the first end 8 to the second end 10 that is located between flat supports 22. The split 12 allows the securement device 2 to flex open and closed to accommodate a range of different sized rectal tube 4. In some embodiments within the scope of the present disclosure, the securement device 2 has a hollow cylindrical tube 6 sized to accommodate a rectal tube 4 that is approximately 10.7 mm in diameter. As such, the split 12 can have a width w in a range of approximately 1-3 mm wide when undeformed. The rectal tube 4 is snapped into the hollow cylindrical tube 6 through the split 12.

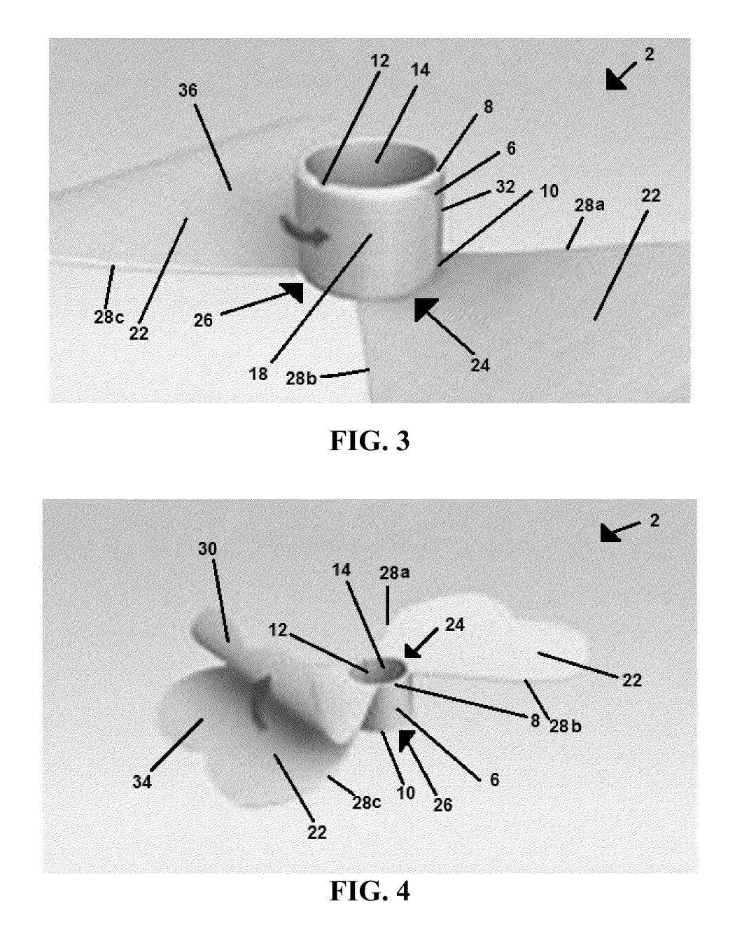

In some embodiments within the scope of the present disclosure, at least a portion of an interior portion 14 of the hollow cylindrical tube 6 is coated with an adhesive that sticks to an outer side of a rectal tube 4 once it has been snapped into the hollow cylindrical tube 6. The adhesive on the interior portion 14 of the hollow cylindrical tube 6 may be covered by a removable protective backing 16 (not depicted). The adhesive may cover all or just a portion of the interior portion 14 of the hollow cylindrical tube 6. In some embodiments within the scope of the present disclosure, in addition to or instead of adhesive on the interior portion 14, an adhesive flap 18 can be secured to the exterior portion 32 of the hollow cylindrical tube 5 on one side of the split 12. Once the rectal tube 4 has been snapped into the hollow cylindrical tube 6 through the split 12, the adhesive flap 18 may be wrapped around the outside of the hollow cylindrical tube 6 across the split 12 and adhered to the hollow cylindrical tube 6 on the opposite side of the split 12 from which the adhesive flap 18 started, as shown in FIG. 3. The adhesive flap 18 may have a removable protective backing 20 that must be removed, as shown in FIG. 2, prior to being adhered on the opposite side of the split 12 from which it started. The adhesive flap 18 compresses the interior 14 of the hollow cylindrical tube 6 onto the exterior of the rectal tube 4 that helps to secure the hollow cylindrical tube 6 to the rectal tube 4.

As best shown in FIGS. 1 and 4, the flat supports 22 are connected to the second end 10 of the hollow cylindrical tube 6 and extend radially outward therefrom. Each flat support 22 has a proximal surface 34 located against the patient's skin when the securement device 2 is properly positioned and a distal surface 36 on the opposite side of the proximal surface 34. At least a portion of the proximal surface 34 is coated with an adhesive that is secured to the skin of a patient in order to keep the securement device 2, and consequently the rectal tube 4, in place. The adhesive may coat all or only a portion of the proximal surface 34. The flat supports 22 may be made of a flexible material such as a hydrocolloid. The flat supports 22 may be a porous, non-porous, mesh, or non-mesh material. In FIGS. 1-4, two flat supports 22 are disposed on opposite sides of the hollow cylindrical tube 6 and connected to the second end 10 of the hollow cylindrical tube 6 by an adhesive, glue, welding, or by being integrally formed with the hollow cylindrical tube 6. In other embodiments within the scope of the present disclosure, one flat support 22 or more than two flat supports 22 may be connected to the second end 10 of the cylindrical tube 6 by an adhesive, glue, welding, or by being integrally formed with the hollow cylindrical tube 6.

In FIGS. 1-4, the flat supports 22 are bow shaped in order to avoid a situation in which stool that has leaked around the outside of the rectal tube 4 is trapped underneath the flat supports 22. The bow shape allows the flat supports 22 to have a limited length connection 24 to the second end 10 of the hollow cylindrical tube 6, such that at least a portion 26 of the circumference of the second end 10 of the cylindrical tube 6 is not connected to a flat support 22. Stated another way, portion 26 is free from connection to the flat supports 22. In some embodiments, portion 26 constitutes an exposed rim portion of the hollow cylindrical tube 6. The portion 26 allows leaked stool to escape from around the edges of the second end 10 of the hollow cylindrical tube 6 and be wiped away, resulting in a more sanitary and comfortable condition for the patient. In other embodiments, the flat supports 22 could be made of a material that is sufficiently porous to allow leaked fecal matter to pass through the flat supports 22.

The side edges 28 (28a, 28b, 28c, and 28d) of each bow shaped flat support 22 diverge from each other and extend outward from the limited length connection 24 such that each bow shaped flat support 22 has a V-shape. This shape increases the surface area of the flat support 22. The side edges 28a and 28b are disposed at an angle β relative to one another in the V-shape, which corresponds with the length of the limited length connection 24, and the angle β may range from approximately 20 degrees to approximately 140 degrees. In contrast, an angle a between side edges 28a and 28c, which corresponds with the length of portion 26, may range from approximately 40 to approximately 160 degrees. Because the surface area of the flat support 22 includes adhesive and is secured to the skin of the patient, a larger surface area may be better able to stick in place. Although FIGS. 1-4 all depict the flat supports 22 as bow shaped, other embodiments within the scope of the present disclosure may have flat supports with other shapes. The flat supports 22 may have a removable protective backing 30 covering the adhesive that should be removed prior to use. In addition, a no-sting barrier wipe may be provided in connection with the securement device 2 in order to prepare the patient's skin so that it will better adhere to the flat supports 22

In use, a healthcare professional inserts a rectal tube 4 into a patient's anus. The securement device 2 is secured to the rectal tube 4 by snapping a portion of the rectal tube 2 that is near the patient's anus through the split 12 into the hollow cylindrical tube 6. If the hollow cylindrical tube 6 has adhesive on its interior portion, any removable protective backing 16 (not depicted) covering the adhesive should be removed prior to snapping the rectal tube 4 into the hollow cylindrical tube 6 so that the interior portion 14 of the hollow cylindrical tube 6 will immediately adhere to the outer side of the rectal tube 4. If the hollow cylindrical tube 6 has an adhesive flap 18, any removable protective backing 20 covering the adhesive on the adhesive flap 18 is removed and the adhesive flap 18 is then tightly wrapped around the exterior portion 32 of the hollow cylindrical tube 6 and adhered to the hollow cylindrical tube 6 across the split 12 on the opposite side of the split 12 from which it originated. Any removable protective backing 30 covering the adhesive on the flat supports 22 is then removed, and the patient's skin may optionally be wiped with a no-sting barrier wipe to enhance the ability of adhesive to stick to the patient's skin. The flat supports 22 are then adhered to the skin of the patient's buttocks. In this way, the rectal tube 4 is secured to the patient and is not easily dislodged.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A securement device for securing a rectal tube comprising:
a hollow cylindrical tube sized to accommodate a rectal tube, the hollow cylindrical tube having an opening at a first end and at a second end;
a split running in an axial direction from the first end to the second end of the hollow cylindrical tube;
one or more flat supports connected to the second end of the hollow cylindrical tube, each of the one or more flat supports coated with an adhesive that can secure the one or more flat supports to skin of a patient;
wherein each of the one or more flat supports has a limited length connection with the second end of the hollow cylindrical tube such that at least a portion of the circumference of the second end of the hollow cylindrical tube is not connected to a flat support.

2. The securement device of claim 1, wherein the hollow cylindrical tube further comprises a fixation device comprising at least one of (a) and (b):
(a) an adhesive flap secured to an exterior portion of the hollow cylindrical tube on one side of the split and configured to wrap around an exterior portion of the hollow cylindrical tube across the split and adhere to the hollow cylindrical tube on the opposite side of the split from which the adhesive flap originates, and
(b) an adhesive coating on at least a portion of an interior portion of the hollow cylindrical tube.

3. The securement device of claim 1, and the hollow cylindrical tube is made from a flexible, non-latex material.

4. The securement device of claim 1 comprising two flat supports.

5. The securement device of claim 4, wherein the two flat supports are disposed on opposite sides of the hollow cylindrical tube.

6. The securement device of claim 1, wherein each of the one or more flat supports has side edges that are angled relative to one another so as to increase the surface area of the flat support.

7. The securement device of claim 6, wherein each of the one or more flat supports is bow shaped.

8. The securement device of claim 1, wherein each of the one or more flat supports is made of a hydrocolloid.

9. The securement device of claim 2, wherein a removable protective backing is provided for at least one of the following (a) through (c): (a) the adhesive flap, (b) the one or more flat supports, and (c) the interior of the hollow cylindrical tube.

10. A medical device comprising:
a rectal tube; and
a securement device comprising:
(a) a hollow cylindrical tube adapted to attach to a portion of the rectal tube,
(b) a first opening at a first end of the hollow cylindrical tube and a second opening at a second end of the hollow cylindrical tube,
(c) a split running in an axial direction from the first end to the second end of the hollow cylindrical tube, and
(d) one or more flat supports connected to the second end of the hollow cylindrical tube, each of the one or more flat supports at least partially coated with an adhesive that can secure the one or more flat supports to skin of a patient.

11. The medical device of claim 10, wherein each of the one or more flat supports has a limited length connection with the second end of the hollow cylindrical tube such that at least a portion of the circumference of the second end of the hollow cylindrical tube is not connected to a flat support.

12. The medical device of claim 10, wherein the hollow cylindrical tube further comprises a fixation device comprising at least one of (a) and (b):
(a) an adhesive flap secured to an exterior portion of the hollow cylindrical tube on one side of the split and configured to wrap around an exterior portion of the hollow cylindrical tube across the split and adhere to the hollow cylindrical tube on the opposite side of the split from which the adhesive flap originates, and
(b) an adhesive coating on at least a portion of an interior portion of the hollow cylindrical tube.

13. The medical device of claim 10, and at least one of (a) and (b):
(a) the hollow cylindrical tube is made from a flexible, non-latex material, and
(b) each of the one or more flat supports is made of a hydrocolloid.

14. The medical device of claim 10 comprising two flat supports that are disposed on opposite sides of the hollow cylindrical tube.

15. The medical device of claim 10, wherein each of the one or more flat supports has side edges that are angled relative to one another so as to increase the surface area of the flat support.

16. The medical device of claim 15, wherein each of the one or more flat supports is bow shaped.

17. The medical device of claim 12, wherein a removable protective backing is provided for at least one of the following (a) through (c): (a) the adhesive flap, (b) the one or more flat supports, and (c) the interior of the hollow cylindrical tube.

18. A securement device for securing a rectal tube comprising:
a hollow cylindrical tube sized to accommodate a rectal tube, the hollow cylindrical tube having an opening at a first end and at a second end;
a split running in an axial direction from the first end to the second end of the hollow cylindrical tube;
two bow shaped flat supports disposed on opposite sides of the hollow cylindrical tube and connected to the second end of the hollow cylindrical tube, each of the bow shaped flat supports coated with an adhesive that can secure the one or more flat supports to skin of a patient;
wherein each of the two bow shaped flat supports has a limited length connection with the second end of the hollow cylindrical tube such that at least a portion of the circumference of the second end of the hollow cylindrical tube is not connected to a bow shaped flat support;
an adhesive flap secured to an exterior portion of the hollow cylindrical tube on one side of the split and configured to wrap around the exterior portion of the hollow cylindrical tube across the split and adhere to the hollow cylindrical tube on the opposite side of the split from which the adhesive flap originates;
at least a portion of an interior portion of the hollow cylindrical tube coated with an adhesive;
a removable protective backing provided on the adhesive flap;
a removable protective backing provided on each of the two bow shaped flat supports; and
a removable protective backing provided on the interior of the hollow cylindrical tube.

19. The securement device of claim 18, and the hollow cylindrical tube is made from a flexible, non-latex material.

20. The securement device of claim 18, and the two bow shaped flat supports are made of a hydrocolloid.
